(19) United States
(12) Patent Application Publication
(10) Pub. No.: US 2026/0242844 A1
(43) Pub. Date: Aug. 20, 2026

(54) METHOD OF DETECTING TARGET SUBSTANCE, REAGENT, AND IN VITRO DIAGNOSTIC KIT

(71) Applicant: CANON KABUSHIKI KAISHA, Tokyo (JP)

(72) Inventors: FUMIO YAMAUCHI, Kanagawa (JP); NORISHIGE KAKEGAWA, Tokyo (JP); KENJI KITAGAWA, Shiga (JP); TSUYOSHI NOMOTO, Tokyo (JP)

(21) Appl. No.: 19/545,512

(22) Filed: Feb. 20, 2026

(30) Foreign Application Priority Data
Feb. 20, 2025 (JP) 2025-026283

(51) Int. Cl. C12Q 1/48; G01N 21/64

(52) U.S. Cl. CPC C12Q 1/48; G01N 21/6428; G01N 21/6445; G01N 2021/6439; G01N 2333/91085

(57) ABSTRACT

Provided is a fast, simple, high-sensitivity method of detecting a target substance related to the enzyme reaction of a transglutaminase in a liquid sample, a reagent, and an in vitro diagnostic kit. Specifically, provided is a reagent for detecting a transglutaminase through use of a value related to fluorescence anisotropy, the reagent including: a luminescent particle having a first substrate of the transglutaminase; and a plurality of second substrates of the transglutaminase.

### BACKGROUND

### Field of the Technology

The present disclosure relates to a method of detecting a target substance, a reagent, and an in vitro diagnostic kit.

### Description of the Related Art

Enzymes each have a role in catalyzing a chemical reaction in a living body, and the measurement of enzyme activity is extremely important in the fields of not only medical research of enzymes but also inspection of enzymes, clinical inspections utilizing enzymes as reagents, and substance production utilizing enzymes.

A transglutaminase (hereinafter sometimes abbreviated as “TG”) is an enzyme, which uses a glutamine residue in a polypeptide as a substrate, and catalyzes an amide bond-forming reaction between a carboxamide side chain of the glutamine residue and an amino group from a lysine residue, for example. The family of the transglutaminases is formed of 8 enzymes/enzyme precursors referred to as “TG1” to “TG7” and “factor XIII.” It has been known that the transglutaminase has a physiological role, such as blood coagulation by the factor XIII or localized epidermization by the transglutaminase. In recent years, the pathological importance of the transglutaminase in a neurodegenerative disease has been proven, and hence the development of a high-sensitivity measurement technology for transglutaminase has been expected in order to perform drug development, treatment, or diagnosis based on the transglutaminase.

Fluorescence polarization measurement has been known as one of the methods of measuring enzyme activity. The fluorescence polarization measurement, which utilizes fluorescence polarization, is a method of measuring the rotational motion of a fluorescent substance. When the fluorescent substance is excited, the anisotropy of its fluorescence (also referred to as “degree of fluorescence polarization”) changes in accordance with the rotational motion of the fluorescent substance. That is, the method utilizes the fact that when the fluorescent substance does not rotate, polarized luminescence is observed, whereas when the fluorescent substance freely rotates, fluorescence is radiated in all planes, and as a result, the polarization is eliminated. A feature of the fluorescence polarization measurement is that in the assay, the degree of fluorescence polarization is used as an indicator, not the luminescence intensity of the fluorescence. Another feature is that the method does not require a separation or washing operation. That is, a homogeneous assay is possible, and a specimen can be added to a solution and subjected to measurement as is. Thus, no complicated separation work is required, and measurement time can be shortened.

A method of measuring the activity of a transglutaminase with fluorescence has heretofore been disclosed. In International Publication No. WO2018/004014, the activity of a transglutaminase is examined by measuring fluorescence resonance energy transfer between different fluorescent dyes caused by the bonding of two kinds of substrate peptides labeled with the fluorescent dyes based on the activity of the transglutaminase. However, in the measurement method of International Publication No. WO2018/004014, one substrate peptide molecule is labeled with one fluorescent molecule, and hence a signal change amount per one reaction reduces. Accordingly, when the concentration of an enzyme is extremely low, it has become difficult to measure the activity of the enzyme in some cases. In addition, the fluorescent dyes are each susceptible to its surrounding environment, and hence in a liquid sample containing a large amount of contaminants such as blood, the fluorescent dyes each adsorb to, for example, a protein or a lipid serving as a contaminant to reduce the substrate recognizability of the peptide bonded to the fluorescent dye. Accordingly, it has become difficult to perform high-sensitivity measurement in some cases.

U.S. Patent Application Publication No. 2024/0093087 discloses an immunoassay technology using fluorescence polarization measurement. In U.S. Patent Application Publication No. 2024/0093087, a high-sensitivity immunoassay is constructed by using luminescent particles to aggregate the luminescent particles via an antigen-antibody reaction, and by observing a change in degree of fluorescence polarization before and after the aggregation. In U.S. Patent Application Publication No. 2013/0315996, there is a disclosure of a technology of producing a hydrogel with a transglutaminase.

However, in each of the methods described in the above-mentioned patent literatures, it has been difficult to detect a target substance related to the enzyme reaction of a transglutaminase in a liquid sample simply and with high sensitivity.

### SUMMARY

The present disclosure provides that a value related to the fluorescence anisotropy of the amount or activity of an enzyme or a substance related to an enzyme reaction can be obtained in a short time period and with high sensitivity by introducing a mechanism that forms a crosslinked structure through the action of a transglutaminase in a liquid sample to greatly reduce the mobility of a luminescent particle.

That is, according to one aspect of the present disclosure, there is provided a reagent for detecting a transglutaminase through use of a value related to fluorescence anisotropy, the reagent including: a luminescent particle having a first substrate of the transglutaminase; and a plurality of second substrates of the transglutaminase.

In addition, according to another aspect of the present disclosure, there is provided a reagent for detecting an activator of a transglutaminase, an activity inhibitor of the transglutaminase, and a third substrate of the transglutaminase through use of a value related to fluorescence anisotropy, the reagent including: the transglutaminase; a luminescent particle having a first substrate of the transglutaminase; and a plurality of second substrates of the transglutaminase.

In addition, according to another aspect of the present disclosure, there is provided an in vitro diagnostic kit including any one of the above-mentioned reagents.

In addition, according to another aspect of the present disclosure, there is provided a method of detecting a target substance related to an enzyme reaction of a transglutaminase in a liquid sample, the method including: a first step of forming, in the liquid sample containing the transglutaminase, a luminescent particle having a first substrate of the transglutaminase, and a second substrate of the transglutaminase, a crosslinked structure by a plurality of the second substrates and the first substrate that the luminescent particle has; and a second step of obtaining a value related to fluorescence anisotropy of the liquid sample after the first step.

Features of the present disclosure will become apparent from the following description of embodiments with reference to the attached drawings. The following description of embodiments is described byway of example.

### BRIEF DESCRIPTION OF THE DRAWINGS

FIG. 1 is a flowchart providing a method of detecting a target substance according to one aspect of the present disclosure.

FIG. 2 is a schematic diagram for describing a high-sensitivity method of detecting a transglutaminase according to one aspect of an embodiment of the present disclosure.

FIG. 3 is a schematic diagram for describing a method of detecting the inhibitor of a transglutaminase according to one aspect of the embodiment of the present disclosure.

FIG. 4 is a schematic diagram for describing a method of detecting the third substrate of a transglutaminase according to one aspect of the embodiment of the present disclosure.

### DESCRIPTION OF THE EMBODIMENTS

The present disclosure is described in more detail below. The term “detection” as used in the present disclosure is used in a meaning including each of the following cases: to determine the presence or absence of a target substance; to detect the target substance; and to qualitatively and quantitatively determine the amount and activity of the target substance.

### (Value Related to Fluorescence Anisotropy Determined by Fluorescence Polarization Measurement)

The present disclosure is directed to a method of detecting the amount and activity of a transglutaminase enzyme reaction-related substance, such as a transglutaminase, the third substrate of the transglutaminase, the activator of the transglutaminase, or the inhibitor of the transglutaminase, through use of a luminescent particle by obtaining a value related to its fluorescence anisotropy.

Fluorescence polarization measurement is used to analyze the mobility of a fluorescent molecule in a solution. The principle of the fluorescence polarization measurement is described by the case of the luminescent particle of the present disclosure. When a luminescent particle (particle containing a luminescent molecule exhibiting fluorescence anisotropy) in a liquid is excited by plane-polarized light, the particle emits polarized fluorescence in the same plane. However, when the luminescent particle is rotated by its Brownian motion during the excited state, the particle emits fluorescence to a plane different from the excitation plane, and hence its fluorescence anisotropy is eliminated. In other words, the value related to the fluorescence anisotropy represents the degree of rotational motion of the luminescent particle from being excited to emitting fluorescence.

When the luminescent particle in the liquid is singly dispersed in the solution, the particle shows low fluorescence anisotropy because the particle is vigorously rotated by its Brownian motion. Meanwhile, when the viscosity of a dispersion medium for the luminescent particle increases or a large-sized crosslinked structure is bonded to the luminescent particle, the Brownian motion of the luminescent particle in the solution reduces to increase the fluorescence anisotropy. Accordingly, in the fluorescence polarization measurement, the mobility of the luminescent particle in the solution is analyzed by using a change in fluorescence anisotropy thereof as an indicator. A unit “milli P” (hereinafter abbreviated as “mp”) representing a change in plane polarization may be used as an indicator of the fluorescence anisotropy.

### (Method of Detecting Target Substance)

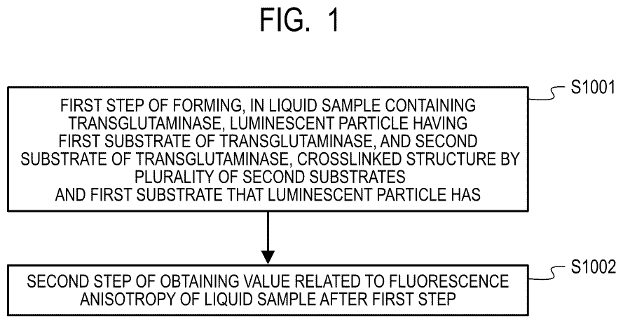

A method of detecting a target substance of the present disclosure is a method of detecting a target substance related to the enzyme reaction of a transglutaminase in a liquid sample, and includes the following steps (1) and (2) as illustrated in FIG. 1. The method of detecting a target substance of the present disclosure may include the following step (3).
(1) A first step (S1001) of forming, in the liquid sample containing the transglutaminase, a luminescent particle having the first substrate of the transglutaminase, and the second substrate of the transglutaminase, a crosslinked structure by a plurality of the second substrates and the first substrate that the luminescent particle has;
(2) A second step (S1002) of obtaining a value related to the fluorescence anisotropy of the liquid sample after the first step
(3) A third step of detecting the target substance related to the enzyme reaction of the transglutaminase in the liquid sample based on the value related to the fluorescence anisotropy.

According to the present disclosure, the high-sensitivity method of detecting a target substance related to the enzyme reaction of a transglutaminase in a liquid sample has the following features: (i) the luminescent particle is used, and hence a signal intensity per molecule is enhanced unlike a conventional low-molecular weight substrate; (ii) an enzyme acts to result in quick formation of the crosslinked structure formed of the second substrate of the transglutaminase (increase in solution viscosity of the liquid sample), and further, the crosslinked structure thus formed crosslinks with the luminescent particle having the first substrate of the transglutaminase, and hence the mobility of the luminescent particle can be significantly suppressed in a short time period, and the value related to the fluorescence anisotropy obtained from the luminescent particle greatly changes in a short time period; and (iii) the measurement of the value related to the fluorescence anisotropy eliminates the need for the separation and washing of a contaminant and an unreacted product, and hence even when the activity of the transglutaminase or a substrate thereof serving as the target substance is low, or the amount thereof is small, the amount and activity of the target substance can be detected with high sensitivity and quickly as compared to an existing method.

### (Measurement Principle 1: Measurement of Transglutaminase Activity)

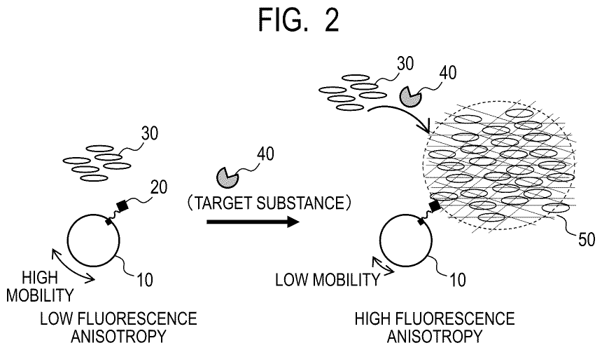

The principle of the present disclosure is described with reference to FIG. 2 by a method of measuring the activity of a transglutaminase.

A transglutaminase 40 serving as a target substance enables the formation of a crosslinked structure formed of a second substrate 30 of the transglutaminase. This is because the activity of the transglutaminase (catalyzing a reaction for forming an amide bond between a glutamine residue in a polypeptide and an amino group) causes crosslinking between the second substrates 30 of the transglutaminase through glutamine and lysine in the second substrate 30 of the transglutaminase. A luminescent particle 10 before the addition of the transglutaminase 40 is dispersed in a liquid sample, and hence its mobility is large. When the transglutaminase 40 is present, a crosslinked structure 50 of the second substrate 30 of the transglutaminase is formed by the crosslinking (also referred to as “aggregation”) between the second substrates 30 of the transglutaminase in accordance with the activity of the transglutaminase, and as a result, the solution viscosity of the liquid sample increases to reduce the mobility of the luminescent particle 10.

Further, the transglutaminase 40 serving as the target substance can crosslink the crosslinked structure 50 of the second substrate 30 of the transglutaminase and the luminescent particle 10 through a first substrate 20 of the transglutaminase present on the surface of the luminescent particle 10. Accordingly, when the transglutaminase 40 is present, the crosslinked structure 50 formed of the second substrate 30 of the transglutaminase and the luminescent particle 10 crosslink with each other in accordance with the activity of the transglutaminase, and as a result, the mobility of the luminescent particle 10 reduces.

A phenomenon in which the mobility of the luminescent particle 10 reduces depending on the activity of the transglutaminase is observed as an increase in fluorescence anisotropy of the luminescent particle 10. Accordingly, the activity of the transglutaminase 40 in a specimen can be determined by obtaining a value related to the fluorescence anisotropy of the luminescent particle 10. For example, the activity of the transglutaminase in the specimen may be determined from the result of the measurement of a value related to the fluorescence anisotropy of the liquid sample based on a calibration curve between a value related to fluorescence anisotropy and activity obtained in advance through use of a transglutaminase whose activity is known.

In the present disclosure, not the activity of the transglutaminase but the amount thereof may be measured. That is, the measurement may be performed by obtaining the value related to the fluorescence anisotropy of the liquid sample dependent on the amount of the transglutaminase while keeping the activity constant.

### (Measurement Principle 2: Measurement of Inhibitor of Transglutaminase)

In the present disclosure, an inhibitor serving as an example of a substance related to the enzyme reaction of a transglutaminase may be subjected to measurement by the same method.

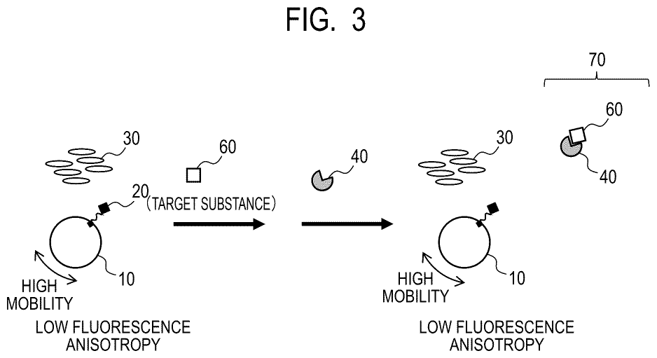

As illustrated in FIG. 3, for example, the inhibition efficiency of an inhibitor 60 of a transglutaminase serving as a target substance on a specified amount of the transglutaminase 40 may be measured by using a value related to the fluorescence anisotropy of the luminescent particle 10 as an indicator.

Specifically, a liquid sample contains a sufficiently large amount of the transglutaminase 40 and/or the transglutaminase 40 having sufficiently high activity. The inhibitor 60 of the transglutaminase serving as the target substance is bonded to the transglutaminase 40 with any affinity to inhibit the activity of the transglutaminase 40. That is, when a transglutaminase 70 having bonded thereto the inhibitor 60 is produced, the activity of the transglutaminase is lost. When the inhibition efficiency is 100%, no crosslinked structure is formed by crosslinking between the second substrates 30 of the transglutaminase, and hence no increase in viscosity of the liquid sample occurs. In addition, no crosslinking reaction between the second substrate 30 of the transglutaminase and the luminescent particle 10 occurs. As a result, the mobility of the luminescent particle 10 does not change, and the fluorescence anisotropy of the luminescent particle 10 also does not change. Meanwhile, when the inhibition efficiency is 0%, a thickening effect on the liquid sample exhibited by such formation of the crosslinked structure 50 of the second substrate 30 of the transglutaminase as illustrated in FIG. 2 and the bonding of the luminescent particle 10 to the crosslinked structure occur. As a result, the fluorescence anisotropy of the luminescent particle 10 increases. That is, a change in value related to the fluorescence anisotropy is observed in accordance with the inhibition efficiency of the inhibitor 60 on the transglutaminase 40. Accordingly, many inhibitor candidates can be simply screened by the detection method of the present disclosure.

### (Measurement Principle 3: Measurement of Activator of Transglutaminase)

In addition to the inhibitor, the activation efficiency of the activator of a transglutaminase may be similarly detected (not shown).

Specifically, a liquid sample contains a small amount of the transglutaminase 40 and/or the transglutaminase 40 having low activity. The activator serving as the target substance is bonded to the transglutaminase 40 with any affinity to improve the activity of the transglutaminase 40. When the activation efficiency is 0%, no crosslinked structure is formed by crosslinking between the second substrates 30 of the transglutaminase, and hence no increase in viscosity of the liquid sample occurs. In addition, no crosslinking reaction between the second substrate 30 of the transglutaminase and the luminescent particle 10 occurs. As a result, the mobility of the luminescent particle 10 does not change, and the fluorescence anisotropy of the luminescent particle 10 also does not change. Meanwhile, when the activation efficiency is 100%, a thickening effect on the liquid sample exhibited by such formation of the crosslinked structure 50 of the second substrate 30 of the transglutaminase as illustrated in FIG. 2 and the bonding of the luminescent particle 10 to the crosslinked structure occur. As a result, the fluorescence anisotropy of the luminescent particle 10 increases. That is, a change in value related to the fluorescence anisotropy is observed in accordance with the activation efficiency of the activator on the transglutaminase 40. Accordingly, many activator candidates can be simply screened by the detection method of the present disclosure.

### (Measurement Principle 4: Measurement of Third Substrate of Transglutaminase)

In addition, the third substrate of a transglutaminase may be subjected to measurement in the same manner as in the inhibition efficiency in the measurement principle 2.

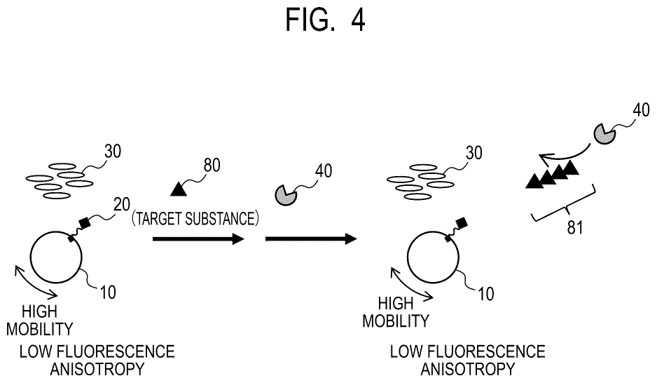

As illustrated in FIG. 4, for example, the inhibition efficiency of a third substrate 80 of a transglutaminase serving as a target substance on a specified amount of the transglutaminase 40 may be measured by using a value related to the fluorescence anisotropy of the luminescent particle 10 as an indicator. The term “inhibition” as used herein means that the third substrate 80 competitively inhibits the reaction of the transglutaminase 40 with each of the first substrate 20 and the second substrate 30.

Specifically, a liquid sample contains a sufficiently large amount of the transglutaminase 40 and/or the transglutaminase 40 having sufficiently high activity. The third substrate 80 of the transglutaminase serving as the target substance independently can form a crosslinked structure as compared to the first substrate 20 and the second substrate 30 through the activity of the transglutaminase 40 (the third substrate 80 has substrate specificity higher than that of the first substrate 20 or the second substrate 30). That is, when the crosslinked structure 81 of the third substrate 80 is formed, the activity of the transglutaminase 40 on the first substrate 20 is inhibited. When the inhibition efficiency is 100%, no crosslinked structure is formed by crosslinking between the second substrates 30 of the transglutaminase, and hence no increase in viscosity of the liquid sample occurs. In addition, no crosslinking reaction between the second substrate 30 of the transglutaminase and the luminescent particle 10 occurs. As a result, the mobility of the luminescent particle 10 does not change, and the fluorescence anisotropy of the luminescent particle 10 also does not change. Meanwhile, when the inhibition efficiency is 0%, a thickening effect on the liquid sample exhibited by such formation of the crosslinked structure 50 of the second substrate 30 of the transglutaminase as illustrated in FIG. 2 and the bonding of the luminescent particle 10 to the crosslinked structure occur. As a result, the fluorescence anisotropy of the luminescent particle 10 increases. That is, a change in value related to the fluorescence anisotropy is observed in accordance with the inhibition efficiency of the third substrate 80 on the transglutaminase 40. Accordingly, many transglutaminase substrate candidates can be simply screened by the detection method of the present disclosure.

### (Specific Description of Detection Method)

An example of the present disclosure includes the following steps (1) and (2). The method of detecting a target substance of the present disclosure may include the following step (3).
(1) A first step of forming, in the liquid sample containing the transglutaminase, a luminescent particle having the first substrate of the transglutaminase, and the second substrate of the transglutaminase, a crosslinked structure by a plurality of the second substrates and the first substrate that the luminescent particle has
(2) A second step of obtaining a value related to the fluorescence anisotropy of the liquid sample after the first step
(3) A third step of detecting the target substance related to the enzyme reaction of the transglutaminase in the liquid sample based on the value related to the fluorescence anisotropy.

Each step is described in more detail below.

### (First Step)

In the present disclosure, the first step is a step in which the crosslinked structure is formed by the plurality of the second substrates of the transglutaminase in the liquid sample, the luminescent particle is bonded to the crosslinked structure thus formed, and the viscosity of the liquid sample increases (also referred to as “thickening”).

Specifically, in the first step, the first substrate of the transglutaminase and the crosslinked structure formed of the second substrate of the transglutaminase can be crosslinked by the transglutaminase. Further, in the first step, the viscosity of the liquid sample can be increased by the crosslinked structure formed of the second substrate of the transglutaminase. Only the thickening of the liquid sample out of those events may occur, only the bonding of the luminescent particle to the crosslinked structure may occur, or both the events may occur.

When the target substance is the transglutaminase, the transglutaminase, the luminescent particle having the first substrate of the transglutaminase, and the second substrate of the transglutaminase are caused to react with each other to provide a bonded product of the formed crosslinked structure and the luminescent particle. Alternatively, the viscosity of the liquid sample is increased. In this case, the order of the reactions is not particularly limited. That is, the transglutaminase, the luminescent particle having the first substrate of the transglutaminase, and the second substrate of the transglutaminase may be simultaneously caused to react with each other, or the following may be performed: the transglutaminase and the second substrate of the transglutaminase are caused to react with each other first to form a crosslinked structure; and in an environment in which transglutaminase activity is maintained, the luminescent particle is caused to react with the crosslinked structure to provide the bonded product of the luminescent particle and the crosslinked structure. The transglutaminase, the luminescent particle having the first substrate of the transglutaminase, and the second substrate of the transglutaminase can be simultaneously reacted. This is because the crosslinked structure in the liquid sample may cause a reduction in dispersibility of the luminescent particle or a reduction in efficiency with which the luminescent particle and the crosslinked structure react with each other. At this time, in the reaction, the contents of the liquid sample are suitably mixed at the temperature at which the transglutaminase is activated (e.g., 30° C. or more and 50° C. or less) and the pH at which the transglutaminase is activated (e.g., a pH of 5 or more and 8 or less).

When the target substance is the inhibitor of the transglutaminase serving as an example of an enzyme reaction-related substance, the transglutaminase may be added to a liquid sample containing the inhibitor, the luminescent particle having the first substrate of the transglutaminase, and the second substrate of the transglutaminase, and caused to react with the contents. In this case, although the order of the reactions is not particularly limited, the inhibitor serving as the target substance can be added in advance of the transglutaminase because the transglutaminase serves as a trigger to cause a crosslinking reaction through a substrate. The liquid sample may be divided into a plurality of solutions.

That is, the following may be performed: a solution A obtained by mixing the transglutaminase and the inhibitor serving as the target substance is prepared; and then the solution A is mixed into a solution B containing the luminescent particle having the first substrate of the transglutaminase and the second substrate of the transglutaminase to perform the bonding of the luminescent particle to the crosslinked structure and the thickening of the liquid sample. At this time, even when the target substance is the activator of the transglutaminase or the third substrate of the transglutaminase, the substance can be detected by performing the same operation as that in the case of the inhibitor.

An example of the first step is as follows: the luminescent particle is bonded to the crosslinked structure formed by the transglutaminase in the liquid sample; and the crosslinked structure is produced independently of the luminescent particle in the liquid sample to thicken the solution of the liquid sample. To achieve the foregoing, the first substrate of the transglutaminase and the second substrate of the transglutaminase can be the same material. For example, a luminescent particle having immobilized thereonto casein serving as the first substrate and casein serving as the second substrate may be used. The mobility of the luminescent particle can be significantly reduced by the actions of those, and as a result, the fluorescence anisotropy of the luminescent particle significantly increases. The detection sensitivity of the target substance can be improved by obtaining a large amount of change in value related to the fluorescence anisotropy.

### (Liquid Sample)

The liquid sample that may be used in the present disclosure may be any liquid (also referred to as “specimen”) that contains a component for measuring the activity and concentration of the target substance, and also contains the target substance. In the present disclosure, the target substance is a substance related to the enzyme reaction of the transglutaminase. For example, a body fluid, such as blood, urine, or saliva, containing the target substance, a buffer solution containing the target substance, a culture liquid or tissue extract of cells or microorganisms containing the target substance, drinking water containing the target substance, river water containing the target substance, or a waste liquid containing the target substance may be used as the specimen.

### (Transglutaminase)

The target substance in the present disclosure is, for example, a transglutaminase that is an enzyme. The transglutaminase uses a glutamine residue in a polypeptide as a substrate, and catalyzes an amide bond-forming reaction between a carboxamide side chain of the glutamine residue and an amino group that a lysine residue has. In the present disclosure, 8 enzymes/enzyme precursors referred to as “TG1” to “TG7” and “factor XIII” may each be used as the transglutaminase.

### (Substance Related to Enzyme Reaction)

A substance related to an enzyme reaction serving as another example of the target substance in the present disclosure is a substance related to a transglutaminase enzyme reaction, and only needs to be a substance whose amount and activity can be measured by the detection method of the present disclosure. That is, the substance related to the enzyme reaction only needs to be a substance that affects the activity of the transglutaminase. The substance is, for example, the third substrate, activator, or inhibitor of the transglutaminase. The value related to the fluorescence anisotropy obtained by the fluorescence polarization measurement of the present disclosure can be changed in accordance with the amount and activity of each of those enzyme reaction-related substances. Examples of the third substrate of the transglutaminase include casein, gelatin, collagen, keratin, modified bodies of each thereof, and partial peptides of each thereof. In particular, a compound that is subjected to catalytic activity by the transglutaminase as compared to the first substrate and second substrate of the transglutaminase is suitable. The activator is, for example, calcium chloride, and the inhibitor is, for example, a metal chelator or a protein modifier. In the present disclosure, the transglutaminase is included in the concept of the substance related to the enzyme reaction of the transglutaminase because the enzyme reaction occurs by virtue of the presence of the transglutaminase.

(Luminescent Particle having First Substrate of Transglutaminase)

The luminescent particle having the first substrate of the transglutaminase to be used in the present disclosure only needs to be the following luminescent particle: the particle contains a luminescent substance, and a value related to the fluorescence anisotropy of its light emission can be obtained by fluorescence polarization measurement. In addition, the luminescent particle according to the present disclosure has the substrate of the transglutaminase (also referred to as “first substrate of the transglutaminase”). Accordingly, a crosslinked structure can be formed between the first substrate of the transglutaminase and the second substrate of the transglutaminase present in the same solution by the action of the transglutaminase. The mode of crosslinking between those substrates may be any bond as long as the bond is catalyzed by the transglutaminase. However, a chemical bond is exemplary, and an amide bond, a peptide bond, an isopeptide bond, or the like is also suitable. The first substrate of the transglutaminase that the luminescent particle has may be incorporated in any state as long as the substrate is recognized by the transglutaminase present in the same solution. However, the substrate can be present on the surface of the luminescent particle.

To form an amide bond, the first substrate of the transglutaminase in the luminescent particle can have at least one glutamine residue and at least one lysine residue on its surface. In this case, examples of the first substrate of the transglutaminase to be immobilized onto the luminescent particle include casein, gelatin, collagen, keratin, modified bodies of each thereof, and partial peptides of each thereof. A known method of immobilizing the transglutaminase onto a protein or peptide particle may be used as a method of immobilizing the first substrate of the transglutaminase onto the luminescent particle. Examples thereof include a physical adsorption method and a chemical bonding method, and the first substrate of the transglutaminase can be physically adsorbed to a luminescent particle having a hydrophobic surface. In addition, the first substrate of the transglutaminase can be bonded to a luminescent particle having a reactive functional group through a chemical bond. For example, an amide coupling method including using a water-soluble carbodiimide is suitable.

In the luminescent particle of the present disclosure, the average particle diameter that is the average of the diameters of the particles can be a diameter of 1 nm or more and 1,000 nm or less, exemplarily 25 nm or more and 500 nm or less, and the average particle diameter can be more exemplary 50 nm or more and 300 nm or less. When the average particle diameter is more than 500 nm, there is a possibility that fluorescence anisotropy before the enzyme reaction, that is, when the luminescent particle is present in a monodisperse state increases, and hence the amount of change in value related to fluorescence anisotropy after bonding between the luminescent particle and the crosslinked structure or after the thickening of the solvent of the solution reduces after the enzyme reaction. In addition, when the average particle diameter is less than 25 nm, the content of the luminescent substance in the particle decreases, and the luminescence intensity per particle decreases. As a result, detection sensitivity and accuracy may decrease, and hence a luminescent particle of 50 nm can be used. The size of the luminescent particle may be examined by dynamic light scattering (DLS) measurement.

As a particle material (matrix material) for the luminescent particle, for example, a nanoparticle of a synthetic polymer, such as polystyrene or polymethacrylate, an inorganic nanoparticle, such as silica, titanium oxide, or iron oxide, or a natural polymer, such as dextran or casein, may be used. Of those, a synthetic polymer can be used as the particle material for the luminescent particle according to the present disclosure because the synthetic polymer has a specific gravity that allows dispersion in water, is stable, and has high productivity. A synthetic polymer is, for example, polystyrene particle that is excellent in particle size controllability.

In addition, the surface of the particle can be hydrophilized. This is because when the surface of the particle is hydrophilized, non-specific aggregation between the luminescent particles can be suppressed, and non-specific adsorption of the luminescent particle to a measurement container (e.g., a plastic cuvette or quartz glass) can also be suppressed. In the detection method using the fluorescence polarization measurement of the present disclosure, a change in mobility of the luminescent particle irrespective of the activity and amount of an enzyme, and the concentration of an enzyme reaction-related substance causes a reduction in accuracy or precision of the detection of the target substance. Accordingly, it is required to avoid non-specific aggregation between the luminescent particles and non-specific adsorption of the luminescent particle to the container. For this reason, the surface of the particle can be coated with a hydrophilic polymer. For example, a polymer, such as polyvinylpyrrolidone, polyethylene glycol, polyhydroxymethacrylate, or dextran, is suitably used. As described above, appropriate introduction of a crosslinkable functional group into the surface of the luminescent particle enables the chemical bonding of the first substrate of the transglutaminase to the particle. For example, when the surface of the luminescent particle has a reactive functional group, such as a thiol group, an amino group, a carboxy group, or a maleimide group, the first substrate of a transglutaminase having a thiol group, a carboxy group, an amino group, or the like can be bonded to the particle. Accordingly, the surface of the luminescent particle is, for example, a surface, which is coated with a hydrophilic polymer such as polyvinylpyrrolidone, and into which a carboxy group for chemically bonding the first substrate of the transglutaminase to the particle is introduced.

The luminescent particle to be used in the present disclosure is characterized by containing a luminescent substance. In this case, the luminescent substance may be any substance that can be incorporated into a particle and can be measured by fluorescence polarization measurement. The luminescent substance may be incorporated into the particle surface or the inside of the particle, and is exemplary incorporated into the inside of the particle. This is because an interaction between the luminescent substance and a contaminant or a water molecule in a solution may occur on the particle surface to affect the dispersibility of the luminescent particle and the obtainment of the value related to the fluorescence anisotropy. Examples of the luminescent substance include a fluorescent dye, such as a fluorescein derivative, a rhodamine derivative, or a cyanine derivative, and a rare earth luminescent complex, such as a europium complex or a terbium complex. The europium complex is suitable as the luminescent substance to be incorporated into the luminescent particle according to the present disclosure because its emission wavelength and intensity are not easily affected by the surroundings and its emission lifetime is long.

Specific examples of the europium complex include tris(2-thenoyltrifluoroacetone)bis(triphenylphosphineoxide)europium(III), tris(2-thenoyltrifluoroacetone)(triphenylphosphineoxide)(dibenzylsulfoxide)europium(III), and tris(2-thenoyltrifluoroacetone)(phenanthroline)europium(III).

The content of the luminescent substance in the luminescent particle can be higher. This is because the luminescence intensity per particle increases, and as a result, the increase can contribute to higher sensitivity or improved measurement accuracy of the measurement of the activity and amount of an enzyme, and the concentration of a substance related to an enzyme reaction of the present disclosure. The content of the rare earth luminescent complex in the luminescent particle can be, for example, 0.001 g or more of the rare earth luminescent complex per 1 g of the particle. With this content, the luminescence intensity per particle is sufficiently high and stable luminescence can be obtained. In addition, the content of the luminescent substance may be measured by elemental analysis using the specific element of the luminescent substance as an indicator, and for example, the content of the rare earth luminescent complex may be calculated from the quantification of the rare earth luminescent complex by inductively coupled plasma (ICP) emission measurement.

### (Second Substrate of Transglutaminase)

In the present disclosure, the second substrate of the transglutaminase is a substrate molecule serving as a raw material for forming a crosslinked structure. In the present disclosure, the second substrate of the transglutaminase can have a role as a precursor of the crosslinked structure. Accordingly, the second substrate of the transglutaminase is not particularly limited as long as the substrate does not inhibit the obtainment of a value related to the fluorescence anisotropy of the luminescent particle to be used in the present disclosure, and crosslinking only needs to be formed between the substrate and another molecule of the second substrate present in the same solution by the action of the transglutaminase. In addition, the second substrate is capable of crosslinking with the first substrate of the transglutaminase. The mode of crosslinking between those substrates may be any bond as long as the bond is catalyzed by the transglutaminase. However, a chemical bond is exemplary, and an amide bond, a peptide bond, an isopeptide bond, or the like is also suitable. The second substrate of the transglutaminase may be in any state as long as the substrate is recognized by the transglutaminase present in the same solution. However, the substrate can be water-soluble. In addition, there may be used a carrier having immobilized thereonto a plurality of the second substrates of the transglutaminase. To form an amide bond, the second substrate of the transglutaminase needs to have at least one glutamine residue and at least one lysine residue on a molecule thereof. In this case, examples of the second substrate of the transglutaminase include casein, gelatin, collagen, keratin, modified bodies of each thereof, and partial peptides of each thereof.

With regard to the second substrate of the transglutaminase, the molecular weight of the substrate may be appropriately selected in consideration of required sensitivity and the operability of a reagent. In an example of the detection method of the present disclosure, there is a need to bond the luminescent particle to the formed crosslinked structure to greatly reduce the mobility of the luminescent particle. Accordingly, the molecular weight of the substrate can be as large as possible. Meanwhile, before the measurement of enzyme activity, the molecular weight is desirably as small as possible from the viewpoint of the handling of the solution. As the molecular weight increases, the viscosity of the solution may increase to cause a problem with the dispensation accuracy thereof. In view of the foregoing, the second substrate of the transglutaminase can be a substance whose molecular weight falls within the range of from about 1,000 to about 5,000,000, exemplarily from about 10,000 to about 1,000,000. In addition, the substrate is desirably free of any inhibitory activity on an enzyme. In view of those requests, for example, casein, gelatin, modified bodies of each thereof, and partial peptides of each thereof can be used as the second substrate of the transglutaminase. The second substrate of the transglutaminase may be the same substance as the first substrate of the transglutaminase described above.

The size of the crosslinked structure for greatly changing the value related to the fluorescence anisotropy of the luminescent particle in accordance with the enzyme activity can be observed by dynamic light scattering (DLS) measurement. For example, when the average particle diameter of the luminescent particles is about 100 nm, the size of the crosslinked structure can be about 1,000 nm. The bonding of the luminescent particles to the crosslinked structure that is about 10 times as large as their average particle diameter means that their volumes change so as to increase by a factor of about 1,000. Accordingly, the mobility of each of the luminescent particles greatly reduces, and hence a large signal change in fluorescence polarization measurement can be obtained. The average particle diameter can increase by a factor of at least 1.5 or more, exemplarily by a factor of 5 or more, or by a factor of 10 or more.

### (Second Step)

In the present disclosure, the second step is a step of obtaining the value related to the fluorescence anisotropy of the liquid sample after the first step. In the present disclosure, a step of measuring a degree of fluorescence polarization or fluorescence anisotropy as an example of the value related to the fluorescence anisotropy is described. The fluorescence polarization measurement may be performed by a known method, and various commercially available measurement apparatus may be used for that purpose. The fluorescence polarization measurement is also called fluorescence depolarization measurement, but in the present specification, the fluorescence polarization measurement and the fluorescence depolarization measurement are synonymous.

In the present disclosure, an enzyme or an enzyme reaction-related substance that is a target substance may be subjected to fluorescence polarization measurement using the mobility of luminescent particles in a sample solution as an indicator. That is, the crosslinked structure is formed in the sample solution in accordance with the amount and activity of the enzyme or the enzyme reaction-related substance, and the luminescent particle is bonded to the crosslinked structure, with the result that the mobility of the luminescent particle reduces. In addition, the formation of the crosslinked structure in the solution thickens the liquid sample, and as a result, the mobility of the luminescent particle reduces. The present disclosure is characterized in that the mobility of the luminescent particle is detected by the fluorescence polarization measurement. As described above, in the fluorescence polarization measurement, for example, a degree of fluorescence polarization or fluorescence anisotropy may be used as an indicator of the mobility. An example in which the degree of fluorescence polarization (mp) is used as an indicator is described below.

A feature of the present disclosure is to use, as a probe, a luminescent particle containing a europium complex, which exhibits polarized luminescence, as a luminescent substance in the inside of the luminescent particle. A slight change in rotational movement of the luminescent particles in a liquid sample may be captured as a change in polarized luminescence characteristic. Specifically, when the mobility of the luminescent particle is greatly reduced by the fixation of the luminescent particle to the crosslinked structure having a size larger than that of the luminescent particle, a reduction in rotational Brownian motion of the luminescent particle may be captured with high sensitivity as a change in degree of fluorescence polarization.

The timing of the measurement of the degree of fluorescence polarization may be set appropriately. For example, the degree of fluorescence polarization may be measured after the first step. In this mode, only the fluorescence polarization degree needs to be measured when the reaction is completed, and the operation is simple. For example, the degree of fluorescence polarization may be measured 5 minutes after the target substance has been added to the solution.

Alternatively, the degree of fluorescence polarization of the luminescent particle may be measured before the reaction with the target substance (enzyme or enzyme reaction-related substance) in the first step, and the degree of fluorescence polarization may be set as an initial degree of fluorescence polarization (mp(0)), and then the reaction may proceed and a degree of fluorescence polarization (mp(t)) at a certain time point may be measured again through the bonding of the luminescent particle to the formed crosslinked structure, or the degree of fluorescence polarization may be continuously measured at regular time intervals. That is, by measuring the change in degree of fluorescence polarization over time, the amount of change in degree of fluorescence polarization (Δmp) may be determined from a difference between the degree of fluorescence polarization (mp(t)) at a certain time point and the initial degree of fluorescence polarization (mp(0)). The change in degree of fluorescence polarization occurs rapidly, and for example, a sufficient Δmp is obtained in from about 1 minute to about 10 minutes. A rate of change in degree of fluorescence polarization (dmp/dt) may also be determined.

Measurement conditions for the degree of fluorescence polarization can be, for example, as follows: in a liquid at a temperature of from 1° C. to 50° C., the viscosity of the liquid is from 0.5 mPa·s to 50 mPa·s. When the luminescent particle is a luminescent particle containing a europium complex, the concentration of the luminescent particle is not limited as long as the luminescence of the luminescent particle can be detected, and the concentration may be appropriately selected in accordance with, for example, the kind, amount, and activity of the target substance. The degree of fluorescence polarization can be measured, for example, in a range from 0.0001 mg/mL (equivalent to 0.00001% mass) to 1.0 mg/mL (equivalent to 0.1% mass). The degree of fluorescence polarization can be measured in the range of 0.001 mg/mL (equivalent to 0.0001% mass) to 0.1 mg/mL (equivalent to 0.01% mass) to ensure the luminescence intensity from the luminescent particle and avoid the influence of the scattering of the luminescent particle.

### (Third Step)

In the present disclosure, the third step is a step of detecting a substance related to an enzyme reaction in the liquid sample based on the value related to the fluorescence anisotropy of the luminescent particle in the liquid sample obtained in the second step. More specifically, the third step is a step of associating a change in value related to the fluorescence anisotropy with the amount and activity of an enzyme or a substance related to an enzyme reaction.

In the second step, the viscosity of the liquid sample or the extent to which the mobility of the luminescent particle reduces in accordance with its interaction with the crosslinked structure is detected as a value related to the fluorescence anisotropy. The extent to which the mobility of the luminescent particle reduces depends on the amount and activity of the transglutaminase or the substance related to the enzyme reaction present in the liquid sample. Accordingly, the amount and activity of the transglutaminase or the substance related to the enzyme reaction in the liquid sample can be measured by associating the value related to the fluorescence anisotropy, the value being obtained in the second step, with the amount and activity of the transglutaminase or the substance related to the enzyme reaction in the liquid sample to each other. For example, a case in which the value related to the fluorescence anisotropy, the value being obtained in the second step, is high means that the liquid sample is thickened by the formation of the crosslinked structure therein or the luminescent particle is strongly bonded to the crosslinked structure. That is, it can be concluded from the foregoing that the activity of the transglutaminase is high.

Quantitative association may be performed by, for example, determining a relational formula between the amount or activity of an enzyme and a value related to fluorescence anisotropy in advance using an enzyme solution having known enzyme activity, and then using this formula to determine the enzyme activity of the target substance from the value related to the fluorescence anisotropy obtained by detecting the target substance. When such association is performed, the enzyme activity in the liquid sample can be measured. The enzyme activity measured here may also be determined as an enzyme concentration, a mass, the number of molecules, or the like.

In addition, in the third step, the change in value related to the fluorescence anisotropy of the luminescent particle may be handled relatively. That is, the amount and activity of an enzyme or a substance related to an enzyme reaction may be associated relatively, and for example, the value of the degree of fluorescence polarization (mp) obtained by fluorescence polarization measurement may be used. For example, in applications for screening enzymes each having high activity, the screening may be performed simply based on the value of the degree of fluorescence polarization (mp) or the value of the amount of change thereof (Δmp) for an enzyme population of the same concentration.

### (Reagent for Detection)

According to the present disclosure, there can be provided a reagent for use in the method of detecting a target substance related to the enzyme reaction of a transglutaminase in the present disclosure, the reagent including: a luminescent particle having the first substrate of the transglutaminase; and the second substrate of the transglutaminase.

One specific example is a reagent for detecting a transglutaminase through use of a value related to fluorescence anisotropy, the reagent including: a luminescent particle having a first substrate of the transglutaminase; and a plurality of second substrates of the transglutaminase.

Another example is a reagent for detecting a third substrate of a transglutaminase through use of a value related to fluorescence anisotropy, the reagent including: the transglutaminase; a luminescent particle having a first substrate of the transglutaminase; and a plurality of second substrates of the transglutaminase.

Yet another example is a reagent for detecting an activator of a transglutaminase or an inhibitor of the transglutaminase through use of a value related to fluorescence anisotropy, the reagent including: the transglutaminase; a luminescent particle having a first substrate of the transglutaminase; and a plurality of second substrates of the transglutaminase.

The luminescent particle having the first substrate of the transglutaminase, the transglutaminase, and the second substrate of the transglutaminase for forming the reagent, and the third substrate of the transglutaminase, the activator of the transglutaminase, or the inhibitor of the transglutaminase are each exemplary provided in a solution state. However, the materials may each be provided in, for example, a dry state, a frozen state, or a lyophilized state. The reagent in a dry state may be subjected to measurement by being brought into a solution state with a dissolving liquid attached thereto before the measurement. In addition, the reagent of the present disclosure may be a single reagent, or may be divided into a plurality of reagents, such as a first liquid, a second liquid, and a third liquid.

The amount (concentration) of the luminescent particle having the first substrate of the transglutaminase to be incorporated into the reagent in the present disclosure is, for example, from 0.000001% by mass to 20% by mass, more specifically from 0.0001% by mass to 1% by mass. With regard to the concentration of the luminescent particle described in the present disclosure, 1% by mass corresponds to 10 mg/mL. The reagent according to the present disclosure may include a substance, such as a solvent or a blocking agent, in addition to the configuration according to the present disclosure to the extent that the object of the present disclosure can be achieved.

The substances, such as a solvent and a blocking agent, may be incorporated in combination thereof. Examples of the solvent to be used in the present disclosure include various buffer solutions, such as a phosphate buffer solution, a glycine buffer solution, a Good buffer solution, a Tris buffer solution, and an ammonia buffer solution. However, the solvent to be incorporated into the reagent in the present disclosure is not limited thereto.

### (In Vitro Diagnostic Kit)

According to the present disclosure, there can also be provided an in vitro diagnostic kit including the above-mentioned reagent. The in vitro diagnostic kit may include a standard solution, a positive control, a negative control, a serum diluent, or the like in addition to the above-mentioned reagent. The standard solution is a solution of the target substance whose concentration is known. In addition to serum or physiological saline free of the target substance that may be detected, a solvent may be used as the medium of the positive control or the negative control.

### EXAMPLES

The present disclosure is described in more detail below by way of Examples.

### (Luminescent Particles S1: Production of Casein-immobilized Luminescent Particles)

The luminescent particle to be used in the method of measuring the amount and activity of an enzyme or an enzyme reaction-related substance according to the present disclosure is characterized by having the first substrate of the transglutaminase on its surface. A synthesis example of luminescent particles each having casein serving as the first substrate of the transglutaminase on its surface is described below.

First, polyvinylpyrrolidone (PVP-K30: manufactured by Tokyo Chemical Industry Co., Ltd.) was dissolved in a 2-morpholinoethanesulfonic acid (MES) buffer solution (manufactured by Kishida Chemical Co., Ltd.) having a pH of 7 to prepare a solvent A. Subsequently, [tris(2-thenoyltrifluoroacetone)bis(triphenylphosphineoxide)europium(III)](manufactured by Central Techno Corporation, hereinafter abbreviated as “Eu(TTA)3(TPPO)2”) serving as a europium complex, a styrene monomer (manufactured by Kishida Chemical Co., Ltd.), and 3-methacryloxypropyltrimethoxysilane (manufactured by Tokyo Chemical Industry Co., Ltd., hereinafter abbreviated as “MPS”) were mixed to prepare a reaction liquid B. The reaction liquid B was added into a four-necked flask containing the solvent A, and the mixture was stirred with a mechanical stirrer set to 300 rpm. After 15 minutes of stirring under a nitrogen flow condition, the temperature of an oil bath that had been prepared was set to 70° C., and the nitrogen flow was performed for an additional 15 minutes. After the mixed liquid had been heated and stirred, an aqueous solution having dissolved therein potassium persulfate (hereinafter abbreviated as “KPS”) (manufactured by Sigma-Aldrich) was added into the reaction solution, and emulsion polymerization was performed for 20 hours. After the polymerization reaction, the resultant suspension was subjected to ultrafiltration with about 4 L of ion-exchanged water through use of an ultrafiltration membrane having a molecular weight cutoff of 100K to wash the product, to thereby provide a dispersion of luminescent particles.

An aliquot of the dispersion of the luminescent particles obtained by the emulsion polymerization was taken and added to an aqueous solution having dissolved therein 1% by mass of Tween 20 (manufactured by Kishida Chemical Co., Ltd.). After 10 minutes of stirring, a silane coupling agent, X12-1135 (manufactured by Shin-Etsu Chemical Co., Ltd.), was added, and the mixture was stirred overnight (a mass ratio among the particles, pure water, and X12-1135 loaded was 1:300:2). After the stirring, the dispersion was centrifuged, the supernatant was removed, and the precipitate was redispersed with pure water. The operations of centrifugation and redispersion were performed three or more times to wash the product. The precipitate after the washing was redispersed in pure water. Thus, luminescent particles 1 were obtained.

Next, a 0.25 mL aliquot of the particle dispersion of the luminescent particles 1 (particle concentration: 1.2% by mass) was taken, and the solvent was replaced with 1.6 mL of a MES buffer solution having a pH of 6.0. To the MES buffer solution in which the luminescent particles were dispersed, 1-[3-(dimethylamino)propyl]-3-ethylcarbodiimide and N-hydroxysulfosuccinimide sodium salt were added at 0.5% by mass, and the mixture was subjected to a reaction at 25° C. for 1 hour. After the reaction, the dispersion was washed with phosphate buffered saline (hereinafter abbreviated as “PBS”) having a pH of 7.4. Further, casein (derived from milk, manufactured by Wako Pure Chemical Industries, Ltd.) was added to the washed product, and the mixture was subjected to a reaction at 25° C. for 2 hours so that casein was introduced into the surface of each of the particles. After the introduction, the luminescent particles were washed with a Tris buffer solution having a pH of 8.

After that, the luminescent particles were washed with PBS containing 0.01% by mass Tween 20 (hereinafter abbreviated as “PBS-T”) to provide casein-immobilized luminescent particles (hereinafter referred to as “luminescent particles S1”) whose concentration was 1.0% by mass. An aqueous solution containing the luminescent particles S1 is referred to as “solution of the luminescent particles S1.” The concentration of the particles was adjusted by dilution with the PBS-T.

With regard to the fact that casein was introduced into each of the luminescent particles, the presence of casein on the surface of each of the particles was able to be recognized by: adding a BCA reagent (PROTEIN ASSAY BCA KIT, manufactured by Wako Pure Chemical Industries, Ltd.) to the solution of the luminescent particles S1; centrifuging the solution of the luminescent particles S1; then measuring the absorbance (562 nm) of the supernatant; and recognizing that the absorbance increased as compared to that before the addition of the BCA reagent. The sizes of the luminescent particles in the aqueous solution were analyzed by dynamic light scattering (DLS) measurement. As a result, the particles had an average particle diameter of 143.1 nm (z-average particle diameter). The particles had a polydispersity index of 0.019, and hence monodisperse luminescent particles were obtained.

### (Luminescent Particles S2: Production of Gelatin-Immobilized Luminescent Particles)

Luminescent particles each having gelatin serving as the first substrate of the transglutaminase were synthesized in the same manner as in the method of producing the luminescent particles S1 except that casein was changed to gelatin (TYPE A, manufactured by Wako Pure Chemical Industries, Ltd.). The resultant gelatin-immobilized luminescent particles are hereinafter referred to as “luminescent particles S2.”

With regard to the fact that gelatin was introduced into each of the luminescent particles, the presence of gelatin on the surface of each of the particles was able to be recognized by: adding a BCA reagent (PROTEIN ASSAY BCA KIT, manufactured by Wako Pure Chemical Industries, Ltd.) to the solution of the luminescent particles S2; centrifuging the solution of the luminescent particles S2; then measuring the absorbance (562 nm) of the supernatant; and recognizing that the absorbance increased as compared to that before the addition of the BCA reagent. The sizes of the luminescent particles S2 in the aqueous solution were analyzed by dynamic light scattering (DLS) measurement. As a result, the particles had an average particle diameter of 151.3 nm (z-average particle diameter). The particles had a polydispersity index of 0.031, and hence monodisperse luminescent particles were obtained. The particles were increased in sizes as compared to the luminescent particles S1 (casein-immobilized luminescent particles). This is probably because of a difference between the molecular weights of the proteins (because the molecular weight of gelatin is larger than that of casein).

### (Luminescent Particles NC: Production of Tris-Immobilized Luminescent Particles)

Luminescent particles each having Tris were synthesized in the same manner as in the method of producing the luminescent particles S1 except that casein was changed to tris(hydroxymethyl)aminomethane (Tris, manufactured by Wako Pure Chemical Industries, Ltd.). The resultant Tris-immobilized luminescent particles are hereinafter referred to as “luminescent particles NC.” The luminescent particles NC were used as control particles for use in Comparative Examples because the particles were each free of any compound corresponding to the first substrate of the transglutaminase.

The sizes of the luminescent particles NC in the aqueous solution were analyzed by dynamic light scattering (DLS) measurement. As a result, the particles had an average particle diameter of 141.5 nm (z-average particle diameter). The particles had a polydispersity index of 0.007, and hence monodisperse luminescent particles were obtained. The sizes of the particles were smaller than those of the luminescent particles S1 and the luminescent particles S2. This is probably because no protein is immobilized onto each of the particles.

### Example 1 and Comparative Example 1: Fluorescence Polarization Measurement of Transglutaminase with Casein-immobilized Luminescent Particles

The PBS-T, the solution of the luminescent particles S1 (particle concentration: 0.1 mg/mL), and casein serving as a second substrate (final concentration: 8% by mass) were mixed at ratios shown in Table 1 in a 96-well microplate. A transglutaminase (TG-S, manufactured by Ajinomoto Co., Inc.) serving as a target substance was added to the aqueous solution, and the mixture was stirred well (Example 1). In Table 1, the transglutaminase is represented as “TG”. Casein and the transglutaminase were added after having been dissolved in water. As Comparative Example, a sample to which no target substance was added was also prepared. At this time, 10 μL of the PBS-T was added instead of the target substance (Comparative Example 1).

The microwell plate was set in a fluorescence polarization measurement apparatus (Nivo Multimode Microplate Reader). A temperature in the apparatus was adjusted to 37° C. The degree of fluorescence polarization (mp) of the sample solution was measured about 10 minutes after the addition of the target substance. The conditions of the fluorescence polarization measurement apparatus are as follows:
Mode: FP kinetics;
Excitation light: center wavelength of 355 nm/width of 40 nm;
Emission filter: (S) center wavelength of 615 nm/width of 8 nm, (P) center wavelength of 615 nm/width of 8 nm;
Dichroic mirror: D400;
Measurement time: 1,000 ms;
Z-focus: 5 mm;
Measurement spot size: 2 mm on excitation side, 4 mm on emission side;
Flash energy: low (10); and
PMT HV: 1,000.

As shown in Table 1, although the degree of fluorescence polarization of the transglutaminase-free sample (Comparative Example 1) was 92.1, the degree of fluorescence polarization of the transglutaminase-containing sample (Example 1) was as large as 105.6. The foregoing means that in Example 1, the mobility of each of the luminescent particles S1 (casein-immobilized luminescent particles) in the sample solution reduces. Thus, it was found that the bonding of the substrate of each of the luminescent particles S1 to a crosslinked structure (the crosslinked structure of casein serving as the second substrate) was caused by the activity of the transglutaminase. It was found from this Example that the transglutaminase was able to be simply and quickly detected by measuring the degree of fluorescence polarization of the sample solution containing the luminescent particles S1.

### Example 2 and Comparative Example 2: Fluorescence Polarization Measurement of Transglutaminase with Gelatin-Immobilized Luminescent Particles

The PBS-T, the solution of the luminescent particles S2 (particle concentration: 0.1 mg/mL), and casein serving as a second substrate (final concentration: 8% by mass) were mixed at ratios shown in Table 1 in a 96-well microplate, followed by fluorescence polarization measurement in the same manner as in each of Example 1 and Comparative Example 1 (Example 2 and Comparative Example 2). As can be seen from the results of the measurement shown in Table 1, although the degree of fluorescence polarization of the transglutaminase-free sample (Comparative Example 2) was 89.7, the degree of fluorescence polarization of the transglutaminase-containing sample (Example 2) was as large as 104.4. The foregoing means that in Example 2, the mobility of each of the luminescent particles S2 (gelatin-immobilized luminescent particles) in the sample solution reduces. Thus, it was found that the bonding of the substrate of each of the luminescent particles S2 to a crosslinked structure (the crosslinked structure of casein serving as the second substrate) was caused by the activity of the transglutaminase. It was found from this Example that the transglutaminase was able to be simply and quickly detected by measuring the degree of fluorescence polarization of the sample solution containing the luminescent particles S2.

### Comparative Example 3 and Comparative Example 4: Control Tests with Luminescent Particles onto Each of which First Substrate is not Immobilized

The PBS-T, the solution of the luminescent particles NC (particle concentration: 0.1 mg/mL), and casein serving as a second substrate (final concentration: 8% by mass) were mixed at ratios shown in Table 1 in a 96-well microplate. The mixture was subjected to fluorescence polarization measurement in the same manner as in each of Example 1 and Comparative Example 1. As can be seen from the results of the measurement shown in Table 1, the degree of fluorescence polarization of the transglutaminase-containing sample (Comparative Example 3) was 90.6, and the degree of fluorescence polarization of the transglutaminase-free sample (Comparative Example 4) was 91.8, and hence both the samples were comparable in degree of fluorescence polarization to each other. No increase in degree of fluorescence polarization by virtue of the presence of the transglutaminase was recognized unlike Example 1 and Example 2.

TABLE 1

| | | | | | | Final | | |
|---|---|---|---|---|---|---|---|---|
| | | | | | Casein | concen- | | |
| | | Luminescent | Luminescent | Luminescent | 16% | tration | TG | Degree of |
| | | particles S1 | particles S2 | particles NC | by | of casein | Target | fluorescence |
| Stock | PBS-T | (0.1 mg/mL) | (0.1 mg/mL) | (0.1 mg/mL) | mass | (% by | substance | polarization |
| solution | Composition of reagent (μL) | | | | | mass) | (μL) | (mp) |
| Example 1 | 50 | 10 | 0 | 0 | 50 | 8 | 10 | 105.6 |
| Comparative | 50 | 10 | 0 | 0 | 50 | 8 | 0 | 92.1 |
| Example 1 | | | | | | | | |
| Example 2 | 50 | 0 | 10 | 0 | 50 | 8 | 10 | 104.4 |
| Comparative | 50 | 0 | 10 | 0 | 50 | 8 | 0 | 89.7 |
| Example 2 | | | | | | | | |
| Comparative | 50 | 0 | 0 | 10 | 50 | 8 | 10 | 90.6 |
| Example 3 | | | | | | | | |
| Comparative | 50 | 0 | 0 | 10 | 50 | 8 | 0 | 91.8 |
| Example 4 | | | | | | | | |

It is conceivable that under the experimental conditions of each of Examples 1 and 2, and Comparative Examples 1 to 4 described above, the crosslinked structure (crosslinked structure of casein serving as the second substrate) formed in the solution did not cause an increase in viscosity of the solution enough to change the degree of fluorescence polarization of the luminescent particles.

It was found from the results of Examples 1 and 2, and Comparative Examples 1 to 4 described above that the transglutaminase was able to be simply and quickly subjected to measurement by using a reagent for the fluorescence polarization detection of the transglutaminase, the reagent including, as its constituent components, the luminescent particles each having the first substrate of the transglutaminase and a plurality of the second substrate (casein) of the transglutaminase.

### Example 3: Fluorescence Polarization Measurement of Transglutaminase

The PBS-T, the solution of the luminescent particles S1 (particle concentration: 0.1 mg/mL), and casein serving as a second substrate (final concentration: 1% by mass) were mixed at ratios shown in Table 2 in a 96-well microplate. A transglutaminase (TG-S, manufactured by Ajinomoto Co., Inc.) serving as a target substance was added to the aqueous solution, and the mixture was stirred well. In Table 2, the transglutaminase is represented as “TG”. Fluorescence polarization measurement was performed in the same manner as in Example 1 except that the degrees of fluorescence polarization (mp) of the resultant solution were measured at the following three points: before the addition of the target substance (degree of fluorescence polarization before the addition of the transglutaminase); about 3 minutes thereafter (degree of fluorescence polarization 3 minutes after the addition of the transglutaminase); and about 45 minutes thereafter (degree of fluorescence polarization 45 minutes after the addition of the transglutaminase).

### Example 4: Fluorescence Polarization Measurement of Transglutaminase

Fluorescence polarization measurement was performed in the same manner as in Example 3 except that the final concentration of casein serving as the second substrate was changed to 2% by mass as shown in Table 2.

### Example 5: Fluorescence Polarization Measurement of Transglutaminase

Fluorescence polarization measurement was performed in the same manner as in Example 3 except that the final concentration of casein serving as the second substrate was changed to 3% by mass as shown in Table 2.

### Example 6: Fluorescence Polarization Measurement of Transglutaminase

Fluorescence polarization measurement was performed in the same manner as in Example 3 except that the final concentration of casein serving as the second substrate was changed to 4% by mass as shown in Table 2.

### Example 7: Fluorescence Polarization Measurement of Transglutaminase

Fluorescence polarization measurement was performed in the same manner as in Example 3 except that the final concentration of casein serving as the second substrate was changed to 5% by mass as shown in Table 2.

### Example 8: Fluorescence Polarization Measurement of Transglutaminase

Fluorescence polarization measurement was performed in the same manner as in Example 3 except that the final concentration of casein serving as the second substrate was changed to 6% by mass as shown in Table 2.

As shown in Table 2, the degree of fluorescence polarization after the addition of the transglutaminase increased with increasing final concentration of casein serving as the second substrate. In addition, it was found that the degree of fluorescence polarization increased with time. It is conceivable that the transglutaminase led to the formation of a crosslinked structure in the solution, and the luminescent particles were bonded to the crosslinked structure to cause a reduction in mobility of each of the luminescent particles with time.

The increase rates of the degrees of fluorescence polarization 3 minutes and 45 minutes after the addition of the transglutaminase were calculated. Herein, the increase rate (%) of the degree of fluorescence polarization is a value obtained by multiplying a numerical value, which is obtained by dividing the degree of fluorescence polarization 3 minutes or 45 minutes after the addition of the transglutaminase by the degree of fluorescence polarization before the addition of the transglutaminase, by 100.

The increase rate (%) of each of the degrees of fluorescence polarization 3 minutes and 45 minutes after the addition of the transglutaminase increased with increasing final concentration of casein serving as the second substrate. However, a peak was obtained in the sample (Example 7) in which the final concentration of casein was 5% by mass. It is conceivable that under the conditions, the luminescent particles each having the first substrate of the transglutaminase and the crosslinked structure (crosslinked structure of casein serving as the second substrate) formed in the solution were effectively bonded to each other.

TABLE 2

| | | | | | | | | | Increase rate of | |
|---|---|---|---|---|---|---|---|---|---|---|
| | | | | | | | | | degree of | |
| | | | | | | Degree of fluorescence | | | fluorescence | |
| | | | | | | polarization | | | polarization | |
| | | Luminescent | | Final | | | 3 | 45 | 3 | 45 |
| | | particles | | concentra- | | | Minutes | Minutes | Minutes | Minutes |
| | | S1 | Casein | tion of | TG | Before | after | after | after | after |
| | | (0.1 mg/ | 16% | casein | Target | addition | addition | addition | addition | addition |
| Stock | PBS-T | mL) | by mass | (% by | substance | of TG | of TG | of TG | of TG | of TG |
| solution | Composition of reagent (μL) | | | mass) | (μL) | (mp) | (mp) | (mp) | % | % |
| Example | 93.75 | 10 | 6.25 | 1 | 10 | 64.8 | 65.7 | 67.2 | 101.4 | 103.7 |
| 3 | | | | | | | | | | |
| Example | 87.50 | 10 | 12.50 | 2 | 10 | 68.5 | 70.3 | 71.9 | 102.6 | 105.0 |
| 4 | | | | | | | | | | |
| Example | 81.25 | 10 | 18.75 | 3 | 10 | 70.3 | 74.9 | 75.9 | 106.5 | 108.0 |
| 5 | | | | | | | | | | |
| Example | 75.00 | 10 | 25.00 | 4 | 10 | 73.7 | 78.8 | 82.6 | 106.9 | 112.1 |
| 6 | | | | | | | | | | |
| Example | 68.75 | 10 | 31.25 | 5 | 10 | 75.9 | 82.2 | 92.0 | 108.3 | 121.2 |
| 7 | | | | | | | | | | |
| Example | 62.50 | 10 | 37.50 | 6 | 10 | 86.8 | 89.2 | 102.3 | 102.8 | 117.9 |
| 8 | | | | | | | | | | |

The present embodiment includes the following configurations and methods.

### Method 1

A method of detecting a target substance related to an enzyme reaction of a transglutaminase in a liquid sample, the method comprising:
a first step of forming, in the liquid sample containing the transglutaminase, a luminescent particle having a first substrate of the transglutaminase, and a second substrate of the transglutaminase, a crosslinked structure by a plurality of the second substrates and the first substrate that the luminescent particle has;
a second step of obtaining a value related to polarization anisotropy of the liquid sample after the first step; and
a third step of detecting the target substance related to the enzyme reaction of the transglutaminase in the liquid sample based on the value related to polarization anisotropy.

### Method 2

The method of detecting a target substance according to Method 1, wherein the luminescent particle contains a rare earth complex.

### Method 3

The method of detecting a target substance according to Method 1 or 2, wherein the first step is a step of mixing the liquid sample at 30° C. or more and 50° C. or less and pH of 5 or more and 8 or less.

### Method 4

The method of detecting a target substance according to any one of Methods 1 to 3, wherein obtaining the value related to polarization anisotropy in the second step is also performed in the first step.

### Method 5

The method of detecting a target substance according to any one of Methods 1 to 4, wherein the target substance is transglutaminase.

### Method 6

The method of detecting a target substance according to any one of Methods 1 to 5, wherein the first substrate and the second substrate are each independently selected from the group consisting of: casein; gelatin; collagen; keratin; modified bodies of each thereof; and partial peptides of each thereof.

### Method 7

The method of detecting a target substance according to any one of Methods 1 to 6, wherein the first substrate and the second substrate are the same substance.

### Method 8

The method of detecting a target substance according to any one of Methods 1 to 7, wherein the target substance is a third substrate of the transglutaminase.

### Method 9

The method of detecting a target substance according to any one of Methods 1 to 8, wherein the crosslinked structure is a bond selected from the group consisting of: an amide bond; a peptide bond; and an isopeptide bond.

### Method 10

The method of detecting a target substance according to Method 8, wherein the third substrate has substrate specificity higher than that of each of the first substrate and the second substrate.

### Method 11

The method of detecting a target substance according to Method 8 or 10, wherein the first substrate, the second substrate, and the third substrate are each independently selected from the group consisting of: casein; gelatin; collagen; keratin; modified bodies of each thereof; and partial peptides of each thereof.

### Method 12

The method of detecting a target substance according to any one of Methods 1 to 11, wherein the target substance is an activator of the transglutaminase.

### Method 13

The method of detecting a target substance according to any one of Methods 1 to 12, wherein the first step is a step of increasing a viscosity of the liquid sample.

### Method 14

The method of detecting a target substance according to any one of Methods 1 to 13, wherein the value related to polarization anisotropy is either degree of fluorescence polarization or polarization anisotropic property.

### Configuration 15

A reagent for detecting a transglutaminase through use of a value related to polarization anisotropy, the reagent comprising:
a luminescent particle having a first substrate of the transglutaminase; and
a plurality of second substrates of the transglutaminase.

### Configuration 16

A reagent for detecting a third substrate of a transglutaminase through use of a value related to polarization anisotropy, the reagent comprising:
the transglutaminase;
a luminescent particle having a first substrate of the transglutaminase; and
a plurality of second substrates of the transglutaminase.

### Configuration 17

A reagent for detecting either an activator of a transglutaminase or an activity inhibitor of the transglutaminase through use of a value related to polarization anisotropy, the reagent comprising:
the transglutaminase;
a luminescent particle having a first substrate of the transglutaminase; and
a plurality of second substrates of the transglutaminase.

### Configuration 18

The reagent according to any one of Configurations 15 to 17, wherein the value related to polarization anisotropy is either degree of fluorescence polarization or polarization anisotropic property.

### Configuration 19

The reagent according to any one of Configurations 16, wherein the third substrate has substrate specificity higher than that of each of the first substrate and the second substrate.

### Configuration 20

An in vitro diagnostic kit comprising the reagent of Configurations 15 to 19.

The method of detecting a target substance related to the enzyme reaction of a transglutaminase of the present disclosure enables simple and high-sensitivity measurement of an enzyme reaction-related substance.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2025-026283, filed Feb. 20, 2025, which is hereby incorporated by reference herein in its entirety.

What is claimed is:

1. A reagent for detecting a transglutaminase through use of a value related to fluorescence anisotropy, the reagent comprising:
a luminescent particle having a first substrate of the transglutaminase; and
a plurality of second substrates of the transglutaminase.

2. The reagent according to claim 1, wherein the first substrate and the second substrate are each independently selected from the group consisting of: casein; gelatin; collagen; keratin; modified bodies of each thereof; and partial peptides of each thereof.

3. The reagent according to claim 1, wherein the first substrate and the second substrate are the same substance.

4. The reagent according to claim 1, wherein the luminescent particle contains a rare earth complex.

5. An in vitro diagnostic kit comprising the reagent of claim 1.

6. A reagent for detecting an activator of a transglutaminase, an activity inhibitor of the transglutaminase, and a third substrate of the transglutaminase through use of a value related to fluorescence anisotropy, the reagent comprising:
the transglutaminase;
a luminescent particle having a first substrate of the transglutaminase; and
a plurality of second substrates of the transglutaminase.

7. The reagent according to claim 6, wherein the first substrate and the second substrate are each independently selected from the group consisting of: casein; gelatin; collagen; keratin; modified bodies of each thereof; and partial peptides of each thereof.

8. The reagent according to claim 6, wherein the first substrate and the second substrate are the same substance.

9. The reagent according to claim 6, wherein the third substrate has substrate specificity higher than that of each of the first substrate and the second substrate.

10. The reagent according to claim 6, wherein the luminescent particle contains a rare earth complex.

11. An in vitro diagnostic kit comprising the reagent of claim 6.

12. A method of detecting a target substance related to an enzyme reaction of a transglutaminase in a liquid sample, the method comprising:
forming, in the liquid sample containing the transglutaminase, a luminescent particle having a first substrate of the transglutaminase, and a second substrate of the transglutaminase, a crosslinked structure by a plurality of the second substrates and the first substrate that the luminescent particle has; and
obtaining a value related to fluorescence anisotropy of the liquid sample after the first step.

13. The method of detecting a target substance according to claim 12, wherein the first substrate and the second substrate are each independently selected from the group consisting of: casein; gelatin; collagen; keratin; modified bodies of each thereof; and partial peptides of each thereof.

14. The method of detecting a target substance according to claim 12, wherein the first substrate and the second substrate are the same substance.

15. The method of detecting a target substance according to claim 12, wherein the target substance is one of the transglutaminase, an activator of the transglutaminase, an activity inhibitor of the transglutaminase, or a third substrate of the transglutaminase.

16. The method of detecting a target substance according to claim 15, wherein the third substrate has substrate specificity higher than that of each of the first substrate and the second substrate.

17. The method of detecting a target substance according to claim 12, wherein the crosslinked structure is a bond selected from the group consisting of: an amide bond; a peptide bond; and an isopeptide bond.

18. The method of detecting a target substance according to claim 12, wherein the luminescent particle contains a rare earth complex.

19. The method of detecting a target substance according to claim 12, additionally comprising increasing a viscosity of the liquid sample.
